(19) United States
(12) Patent Application Publication
(10) Pub. No.: US 2004/0038216 A1
(43) Pub. Date: Feb. 26, 2004

(54) Method for the structural determination of ligands bound to macromolecular targets by nuclear magnetic resonance

(76) Inventors: Philip J. Hajduk, Mundelein, IL (US); Bruce A. Beutel, Lake Forest, IL (US); Peter J. Dandliker, Gurnee, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/226,758

(22) Filed: Aug. 23, 2002

(51) Int. Cl. C12Q 1/68; G01N 33/53; G06F 19/00; G01N 33/48; G01N 33/50; G01N 31/00; G01N 33/543

(52) U.S. Cl. 435/6; 435/7.1; 436/518; 702/19

(57) ABSTRACT

The present invention pertains to a method for determining the three-dimensional structure of a macromolecule in complex with a ligand for the design of new pharmaceutically active compounds and other chemical entities.

### FIELD OF THE INVENTION

The present invention pertains to a method for determining the three-dimensional structure of a macromolecule in complex with a ligand for the design of new pharmaceutically active compounds and other chemical entities.

### BACKGROUND OF THE INVENTION

The discovery of new drugs to treat various disease states will undoubtedly continue as the average life expectance increases. Many technological advances have provided methods for the discovery of new drugs. One drug discovery method which has dramatically accelerated the discovery of new drugs is structure-based drug design. In particular the study of the molecular interactions that occur between a drug and the protein that elicits a biological response related to a given disease state provides valuable chemical information which then aids researchers in designing drug molecules with improved activity. The determination of the structure of a protein in complex with a ligand provides crucial information about the binding pocket in an activated binding conformation. This information is then used as a basis to optimize the intermolecular interactions between the ligand and the protein in an iterative process of creating new generations of improved molecules that can be used for the development of new drugs. In addition, once the structural requirements necessary for a compound to have activity are known, further improvements in the physical characteristics of the compound (such as solubility and half-life) may be achieved without disrupting the physical associations between the drug and target protein.

Two of the most successful techniques for obtaining structural information on protein in complex with a ligand are X-ray crystallography and NMR (nuclear magnetic resonance) spectroscopy, both of which are known to those skilled in the art of structure-based drug design efforts. There are, however, limitations inherent to each of these techniques that limit their application to many important biological systems. For example, X-ray crystallography requires suitable crystals for use in this procedure, yet the production of crystals of sufficient size and quality suitable for X-ray crystallography continues to be a major impediment to determining protein structures utilizing this technique. Accordingly, in situations where crystallization of a macromolecule in complex with a ligand cannot provide suitable crystals, X-ray crystallography cannot be used.

High-resolution structure determination using NMR spectroscopy also suffers from many disadvantages (Opella, at al., Methods Enzym. 2001, 339, 285-313; Roberts, DDT 2000, 5, 230-240). The greatest limitation is the need to observe, resolve, and assign the many signals that arise from the spectrum of the protein resonances. In practice, this limits the application of NMR structural studies to those targets that have molecular weights less than about 30 kDa. In addition, the structures of many proteins in complex with a ligand cannot be solved due to problems with forming a complex suitable for NMR analysis. These problems can also include solubility limitations of the ligand as well as dynamic phenomena that broaden the resonances of the protein and/or ligand.

One other method commonly used to predict the structure of the macromolecule in complex with a ligand is that of computational means. It is often suggested that if the structure of the macromolecule is known (either from X-ray crystallography or NMR), then the structure of the macromolecule complexed to a ligand can be obtained using computational methods through a series of computational algorithms. These algorithms, however, are often prone to serious errors (Abagyan and Totrov, Curr. Opin. Chem. Biol. 2001, 5, 375-382). First, most docking algorithms require that the binding site on the macromolecule that elicits the desired biological effect upon ligand binding is known. In practice, the bioactive site for the ligand is not always known, nor can this information always be reliably obtained using independent techniques. Second, even if the binding site can be determined, estimating the binding energy of a macromolecule in complex with a ligand is plagued by problems such as proper balancing of electrostatic and van der Waals interactions, interactions formed by bound water molecules, correctly assigning the ionization states of the acidic and basic groups in the binding site, estimating entopic contributions to the binding energy, and other approximations. All of these factors contribute to inaccuracies in defining the structure of the macromolecule in complex with a ligand which can lead to a computational solution for the complex that can be far from reality.

Accordingly, due to these and other disadvantages within each of these methods, there continues to be a need for new methods of determining the three-dimensional structures of macromolecule in complex with a ligand.

### SUMMARY OF THE INVENTION

The present invention provides a method for determining the 3-dimensional structure of a macromolecule in complex with a ligand molecule, also referred to herein as SOS-NMR, which stands for structural information using overhauser effects and selective labeling via nuclear magnetic resonance spectroscopy. The method comprises the steps of: a) treating a selectively labeled macromolecule whose 3-dimensional structure is known as determined by methods known in the art, with a ligand molecule that forms a complex with the selectively labeled macromolecule, wherein a selectively labeled macromolecule in complex with a ligand is formed; b) determining which portions of the ligand are in close proximity to the selectively labeled regions of the macromolecule ligand complex; c) deriving ambiguous and non-ambiguous distance restraints between the selectively labeled macromolecule and the ligand molecule from the measurements; and d) determining the 3-dimensional structure of the complex of the macromolecule and the ligand molecule using the ambiguous and non-ambiguous distance restraints; e) use the information in the design of pharmaceutically active compounds and other chemical entities.

### BRIEF DESCRIPTION OF THE DRAWINGS

FIG. 1 illustrates the use of selective labeling of a target molecule in order to derive distance information on a ligand-target complex.

FIG. 2 illustrates the use of selective labeling of multiple, equivalent sites on a target molecule in order to derive distance information on a ligand-target complex.

FIG. 3 illustrates the aliphatic regions of ¹H-NMR spectra of Bcl-xL A) fully protonated, b) perdeuterated (no ¹H signals), C) perdeuterated except for ¹H-leucine residues, D) perdeuterated except for ¹H-isoleucine residues, E) perdeuterated except for ¹H-arginine residues, and F) perdeuterated except for ¹H-methionine residues. Approximate locations of the amino acid sidechain signals are indicated above the respective spectra.

FIG. 4 illustrates the use of ambiguous distance restraints to determine the structure of a target-ligand complex.

FIG. 5 illustrates the results of saturation transfer difference experiments on the biaryl 3 (A) in the presence of protonated protein; (B) in the presence of fully deuterated protein; and (C) alone in buffer

FIG. 6 illustrates the results of saturation transfer difference experiments on the biaryl 3 (A) in the presence of protonated protein; (B) in the presence of deuterated protein with selective protonation of arginine residues; (C) in the presence of deuterated protein with selective protonation of tyrosine residues; (D) in the presence of deuterated protein with selective protonation of leucine residues; (E) in the presence of deuterated protein with selective protonation of isoleucine residues; (F) in the presence of deuterated protein with selective protonation of methionine residues. The resonances corresponding to the biaryl are indicated.

FIG. 7 illustrates the result of the identification of potential ligand binding sites (magenta spheres) on Bcl-xL using the ambiguous restraint list derived for biaryl 3 (green carbon atoms).

FIG. 8 illustrates the possible orientations of biaryl 3 (yellow carbons atoms) in the binding site of Bcl-xL (gray surface) using the computational algorithm DOCK4.0 (Kuntz, et al., J. Mol. Biol. 1982, 161, 269-288).

FIG. 9 illustrates the orientations of biaryl 3 predicted by the computational algorithm DOCK4.0 (yellow carbon atoms) that are allowed by the ambiguous NOE data of the current invention (E_{NOE}<1.0) compared to the orientation derived from traditional high-resolution NMR spectroscopy (green carbon atoms).

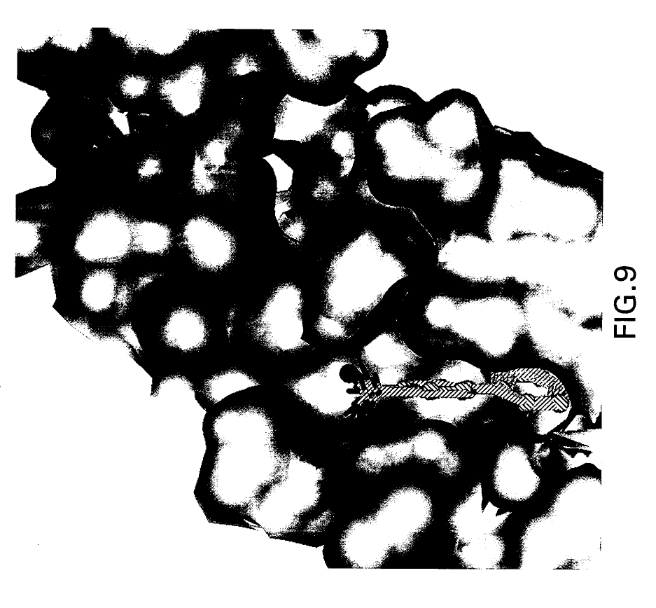

FIG. 10 illustrates a detailed view of the complex between Bcl-xL and biaryl 3.

FIG. 11 illustrates the possible orientations of biaryl 3 (yellow carbon atoms) in the binding site of Bcl-xL using the computational algorithm DOCK4.0 (Kuntz, et al., J. Mol Biol. 1982, 161, 269-288) compared to orientation derived from traditional high-resolution NMR spectroscopy (green carbon atoms). In this case, the protein structure derived from the high-resolution NMR structure of Bcl-xL (gray ribbon) complexed to the Bak peptide (blue ribbon) (Sattler, et al. Science 1997, 275, 983-986) was used for the docking simulations.

FIG. 12 illustrates the orientations of biaryl 3 predicted by the computational algorithm DOCK4.0 (yellow carbon atoms) that are allowed by the ambiguous NOE data of the current invention (E_{NOE}<0.1) compared to orientation derived from traditional high-resolution NMR spectroscopy (green carbon atoms). As in FIG. 11, the protein structure derived from the high-resolution NMR structure of Bcl-xL (gray ribbon) complexed the Bak peptide (blue ribbon) (Sattler, et al. Science 1997, 275, 983-986) was used for the docking simulations.

### DETAILED DESCRIPTION OF THE INVENTION

Definitions

The term “ambiguous distance restraint,” as used herein, refers to a distance restraint between two atoms that are not uniquely identified. For example, the assignment of a restraint that can occur between any atom of type X and any atom of type Y is called an ambiguous restraint.

The term “complex,” as used herein, refers to the combined structure that is formed when a macromolecule and a ligand are combined and form a unique and defined structure.

The term “deuterate” or “deuterated,” as used herein, refers to the incorporation of ²H into a chemical site that would normally be occupied by a proton.

The term “protonate” or “protonated,” as used herein, refers to the incorporation of ¹H into a chemical site that would normally be occupied by a proton.

The term “observables,” as used herein, refers to measureable NMR parameters that exist for a given chemical entity. Examples of NMR observables include, but are not intended to be limited to, chemical shifts, transverse relaxation rates, longitudinal relaxation rates, and nuclear Overhauser effects.

The term “macromolecule,” as used herein, refers to any protein, ribonucleic acid (RNA), deoxyribonucleic acid (DNA), carbohydrate, covalent conjugates, non-covalent oligomers and membrane-associated proteins. Example of covalent conjugates include but are not intended to be limited to, glycoproteins and protein-RNA adducts. Examples of non-covalent oligomers include but are not limited to protein-protein complexes, protein-RNA complexes, protein-DNA complexes. Examples of membrane-associated proteins include, but are not limited to, G-protein coupled receptors (GPCR).

The term “selectively labeled macromolecule,” refers to a molecule that has either the selective incorporation of ²H, ¹³C, ⁵N, or ¹⁹F at specific locations within a macromolecule as defined herein.

The term “unambiguous distance restraint,” as used herein refers to a distance restraint that can occur between two atoms that are uniquely known. For example, the assignment of a distance restraint between a specific known atom X and a specific known atom Y is called an unambiguous distance restraint.

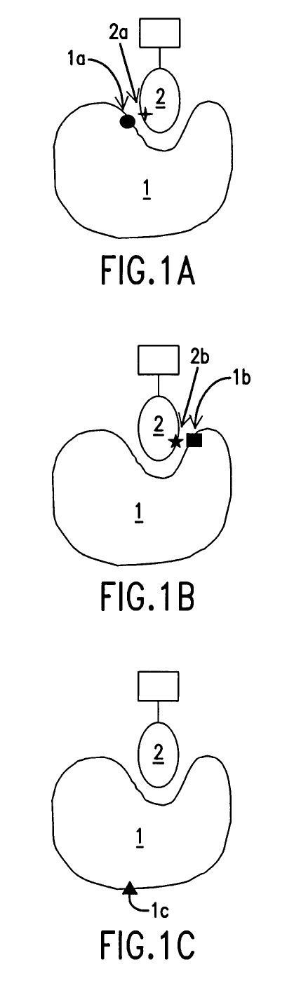

The present invention provides a method for determining the structure of a macromolecule in complex with a ligand. The method relies on performing magnetization transfer experiments between a macromolecule that has been isotopically labeled in specific regions and a ligand to determine whether the regions of the selectively labeled macromolecule are in close spatial proximity to the ligand. By separately labeling different regions of the macromolecule and analyzing the specific portions of the ligand that are in close contact with these regions, a sufficient number of distance restraint can be generated to determine the structure of the macromolecule in complex with the ligand. The information regarding the structure of the macromolecule in complex with the ligand can aid in the development of new ligands that have improved chemical characteristics. This is illustrated in FIG. 1. FIG. 1A shows a single site on target 1 that is labeled (site 1a; shaded oval) and can transfer magnetization to a particular region of ligand 2 (site 2a; four-pointed star). FIG. 1B illustrates an alternate site on target 1 that is labeled (site 1b, shaded square) and can transfer magnetization to a particular region of ligand 2 (site 2b; five-pointed star). FIG. 1C illustrates a third site on target 1 that is labeled (site 1c; shaded triangle) and cannot transfer magnetization to any region of ligand 2 because it is too distant. In this way, if the three-dimensional coordinates of sites 1a, 1b, and 1c are known, a docked orientation of the ligand can be computed.

The method of the present invention utilizes magnetization transfer techniques that are well known in the art. When a ligand binds to a macromolecule, the atoms of the macromolecule and the ligand come into close contact and directly affect each other's local chemical environment. According to the present invention, the relevant interactions between the ligand atoms and the macromolecule atoms are the dipolar couplings between the NMR-active nuclei. NMR-active nuclei are those atoms that have non-zero nuclear spin, the most common nuclei being proton-1 (¹H), carbon-13 (¹³C), nitrogen-15 (¹⁵N), and fluorine-19 (¹⁹F). For example, when a proton of the ligand comes into close proximity (<5 Å) to a proton of the macromolecule, the magnetic fields of each nuclei will affect each other in a time-dependent fashion. If the magnetization state of the macromolecule protons is displaced from equilibrium by a radiofequency pulse, those protons of the ligand that are near the macromolecule will also be perturbed because of the dipolar coupling of the nuclei. These effects are referred to as nuclear Overhauser effects (NOEs). The method of the present invention utilizes these phenomena in that the magnetizations states of the nuclei of the macromolecule are selectively perturbed from equilibrium, which non-equilibrium state then gives rise to changes in the ligand nuclei that are in close proximity to the macromolecule.

There are a variety of methods known in the art to selectively change the magnetization state of a nucleus. One such method involves using selective radiofrequency pulses to irradiate a specific nucleus based on its chemical shift. For example, when a specific proton of the macromolecule exhibits a chemical shift that is well resolved or different from the protons of the ligand (as is typically the case), the specific proton can be selectively irradiated using a selective radiofrequency pulse. Changes in the magnetization of the protons of the ligand due to the irradiation of the protein can then be detected. This technique, called saturation-transfer-difference NMR (STD-NMR), has been used to analyze NOEs between a macromolecule and a ligand, where only specific sites on the ligand that are in close proximity to the macromolecule show NOEs, while parts of the ligand that are distant (>5 Å) from the macromolecule do not show NOEs (Mayer and Meyer, J. Am. Chem. Soc. 2001, 123, 6108-6117). Another method for selectively changing the magnetization state of macromolecule atoms vs. ligand atoms is to isotopically label the macromolecule with ¹³C and then use isotope-editing techniques to selectively invert the magnetization of the macromolecule protons (which are attached to ¹³C) vs. the protons of the ligand (which are not attached to ¹³C). This technique, called NOE pumping, has also been used to detect NOEs between a ¹³C-labeled protein and a ligand (Chen and Shapiro, J. Am. Chem. Soc. 2000, 122, 414-415).

Both STD-NMR and NOE pumping have been successful to identify NOEs between macromolecules and small molecules. However, neither technique provides information about the specific region(s) of the macromolecule that is in close spatial proximity to the ligand. For STD-NMR, this is due to the fact that, although only a small number of macromolecule protons are saturated directly by the selective radiofrequency pulse, all protons on the macromolecule are subsequently saturated due to efficient spin-diffusion processes that occur in large macromolecules. For NOE pumping, the protein is uniformly ¹³C-labeled and therefore all protons are inverted by the isotope-editing sequence. In either case, the NOEs observed to the ligand can originate from virtually anywhere on the protein surface, and it is impossible to obtain detailed proximity information.

The present invention overcomes the limitations presented with other known techniques by incorporating isotopically labeled nuclei at defined positions in the macromolecule in accordance with the teachings of the present invention and as is known in the art. According to one embodiment of the present invention, deuterons (²H) instead of protons (¹H) are incorporated at all positions in the macromolecule except those that are to be probed for NOEs to the ligand. In this case, the NOEs observed in the magnetization transfer experiment can only arise from those positions in the macromolecule that are labeled with ¹H. If the location of the ¹H nuclei on the macromolecule are known (e.g., if the three-dimensional coordinates of the macromolecule are known or can be predicted), then the NOEs that are observed to specific regions of the ligand can be used to define the structure of the macromolecule in complex with a ligand.

Techniques for selective labeling at defined positions within a macromolecule are well known in the art. In particular, preparing protein samples that are deuterated in all positions except for protons at defined amino acid positions have found widespread utility by those skilled in the art of NMR for facilitating the assignment of protein resonances (Vuister, et al., J. Am. Chem. Soc. 1994, 116, 9206-9210; Medek, et al., J. Biomol. NMR 2000, 18, 229-238). In addition, selective protonation and/or deuteration has also been used to probe the interfaces between protein-protein complexes (Takahashi, et al., Nature Struct. Biol., 2000, 7, 220-223) and even to detect NOEs in protein-ligand complexes (Ramesh, et al., Eur. J. Biochem. 1996, 235, 804-813). However, all of these techniques require the observation and assignment of the protein resonances. This requirement limits the applicability of these techniques to those proteins that are of low molecular weight (˜30 kDa) and highly soluble (protein concentrations on the order of 0.5 mM). The method of the present invention overcomes these limitations by not directly observing the protein resonances, but only indirectly observing the effects of magnetization transfer experiments on the ligand resonances. Thus, macromolecules of any size and of limited solubility can be used, as only low concentrations of macromolecule (typically less than 10 μM) are required for the magnetization transfer experiments.

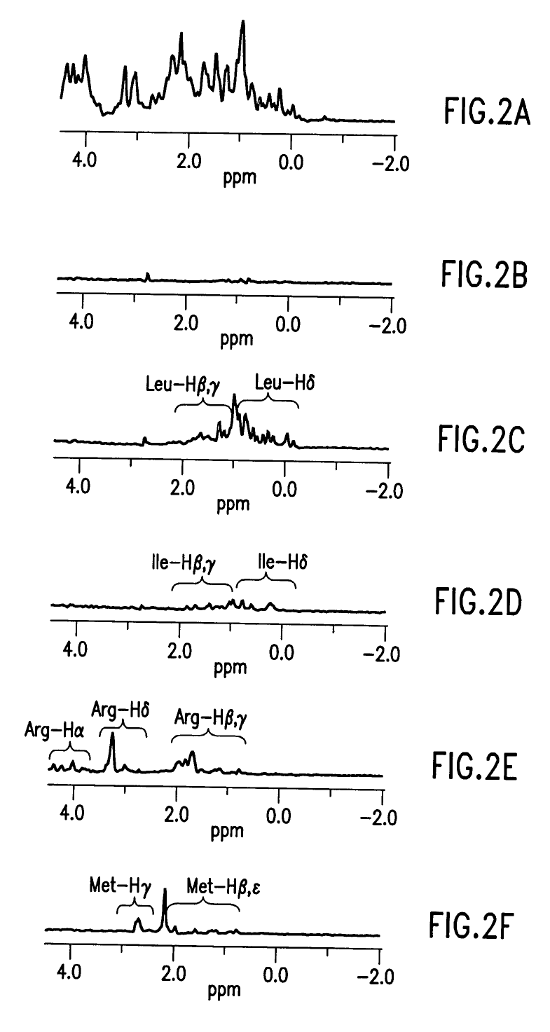

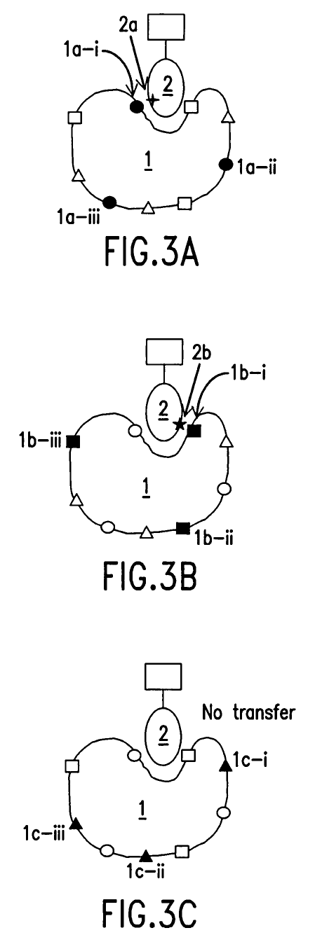

According to the method of the present invention, a series of samples would preferably be prepared, wherein only a single proton site on the macromolecule is labeled with ¹H, while all other protons sites are replaced with ²H (see FIG. 1). Samples of this type would yield unambiguous distance restraints to use in the structure calculation. Alternatively, the incorporation of ¹H's at multiple equivalent sites on the target molecule while deuterating all other sites would give both ambiguous and unambiguous distance restraints. FIG. 2 illustrates the use of selective labeling of multiple, equivalent sites on the target molecule in order to derive distance information on the ligand-target complex. FIG. 2A illustrates three equivalent sites of type “a” on target 1 are labeled (sites 1a-i, 1a-ii, and 1a-iii; shaded ovals). Only the site that is near the ligand (site 1a-i) can transfer magnetization to a particular region of ligand 2 (site 2a; four-pointed star), but this specific site on target 1 is unknown a priori due to the equivalence of the sites. In FIG. 2B, alternate equivalent sites of type “b” on target 1 are labeled (sites 1b-i, 1b-ii, and 1b-iii; shaded squares). Only the site that is near the ligand (site 1b-i) can transfer magnetization to a particular region of ligand 2 (site 2b; five-pointed star), but this specific site on target 1 is unknown a priori due to the equivalence of the sites. In FIG. 2C, three equivalent sites of type “c” on target 1 are labeled (sites 1c-i, 1c-ii, and 1c-iii; shaded triangles). None of these sites are near ligand 2 and therefore no magnetization transfer is detected to any region of 2. Accordingly, if the three-dimensional coordinates of sites 1a, 1b, and 1c are known, the orientation of the ligand can be computed using ambiguous distance restraints. As shown in FIG. 3, one can selectively protonate certain amino acid residues in a protein while incorporating deuterium everywhere else. In such cases, when using such a selectively labeled sample in a magnetization transfer experiment, the NOEs that are observed to the ligand can arise from any amino acid residue of that type. Thus, instead of using specific, unambiguous distance restraints in the structure calculation, one must use ambiguous distance restraints in which the contact defined by the NOE can be between the ligand atom and any of the type-specific amino acid residues in the protein. Such ambiguous restraints are well known in the art and are commonly used in structure calculation programs (see, for example, Linge, et al., Nuclear Magn. Reson. Of Biol. Macromaolecules Pt. B 2001, 339, 71-90). Alternatively, a macromolecule wherein, the incorporation of deuterium at multiple equivalent sites on the target molecule while incorporating protons at all other sites would also give both ambiguous and unambiguous distance restraints. This method would also provide information used to determine the structure of the macromolecule in complex with a ligand and is within the scope of this invention.

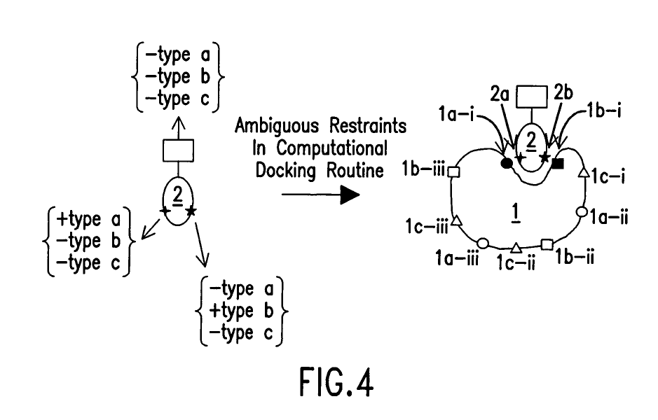

By generating a sufficient number of samples in which different amino acids are selectively labeled, the ambiguous distance restraints can define a unique structure of the protein:ligand complex. This process is illustrated in FIG. 4. First, from the magnetization transfer experiments with selectively labeled target, a restraint list is compiled that delineates which regions of the ligand are in contact with at least one of the multiple, equivalent sites on the target molecule. Next, a computational docking algorithm is applied that generates macromolecule in complex with a ligand that are consistent with the ambiguous distance restraints. For example, the region of ligand 2 denoted with a four-pointed star was determined to be in contact with at least one site of type “a”. Therefore, the docking procedure will require that this portion of the ligand must be in contact either with site 1a-i, and/or 1a-ii, and/or 1a-iii. Similarly, the docking procedure will require that the portion of the ligand denoted with a five-pointed star must be in contact either with site 1b-i, and/or 1b-ii, and/or 1b-iii. No portion of the ligand will be allowed to be in contact with any site of type “c,” as no magnetization transfer was observed between the target and the ligand. The docking procedure will then calculate structures of target-ligand complexes that are consistent with all of the distance information. Preferably, only a single solution is found that satisfies all of the experimental data.

According to one embodiment of the present invention, there is disclosed a method of determining the 3-dimensional structure of a macromolecule in complex with a ligand that comprises the steps of: a) treating a macromolecule whose 3-dimensional structure is known, with a ligand molecule known to form a complex with the macromolecule forming a macromolecule in complex with a ligand; b) performing measurements to determine which portions of the macromolecule are in close proximity to the ligand molecule; c) deriving ambiguous and non-ambiguous distance restraints between the macromolecule and the ligand molecule from the measurements; d) determining the 3-dimensional structure of the macromolecule in complex with the ligand using the ambiguous and non-ambiguous distance restraints. It is to be understood that, according to the present invention, the 3 dimensional structure of the macromolecule is either known or can be determined according to methods known in the art.

The macromolecule used in the method may be one or more samples of the macromolecule in which specific locations have incorporated a ¹H and all other locations have incorporated a deuterium. The macromolecule used in the method may be one or more samples of the macromolecule in which specific locations have incorporated a ²H and all other locations have incorporated a ¹H. Alternatively, the macromolecule used in the method may be one or more samples of the macromolecule in which specific locations have incorporated ¹³C, wherein all other sites are have incorporated ¹²C.

The measurements that determine which portions of the macromolecule are in close proximity to the ligand molecule can involve the use of magnetization transfer experiments to identify NOEs between the selectively labeled regions of the macromolecule and specific positions on the ligand. Furthermore, the magnetization transfer experiments to identify NOEs between the selectively labeled regions of the macromolecule and specific positions on the ligand, may be achieved through saturation difference spectroscopy or selective inversion transfer methods. The macromolecule that is in complex with the ligand can be a polypeptide, RNA, glycoprotein, protein-protein complex, protein-RNA complex, membrane associated protein.

In one embodiment of the present invention, a selectively labeled macromolecule which comprises ¹H incorporated at defined positions whereas all other proton positions comprise ²H is complexed with a ligand, wherein the macromolecule is a polypeptide, RNA, glycoprotein, protein-protein complex, protein-RNA complex, or membrane associated protein. The the complex is assessed by the perturbations in the NMR observables of the ligand as a result of the formation of the selectively labeled macromolecule in complex with the ligand using saturation transfer difference spectroscopy. The the assessments are used to derive ambiguous and non-ambiguous distance restraints used to determine the 3-dimensional structure of the the selectively labeled macromolecule in complex with the ligand.

Alternatively in another embodiment of the present invention, a selectively labeled macromolecule which comprises ²H incorporated at defined positions whereas all other proton positions comprise ¹H is complexed with a ligand, wherein the macromolecule is a polypeptide, RNA, glycoprotein, protein-protein complex, protein-RNA complex, or membrane associated protein. The the complex is assessed by the perturbations in the NMR observables of the ligand as a result of the formation of the selectively labeled macromolecule in complex with the ligand using saturation transfer difference spectroscopy. The the assessments are used to derive ambiguous and non-ambiguous distance restraints used to determine the 3-dimensional structure of the the selectively labeled macromolecule in complex with the ligand.

In another embodiment of the present invention, a selectively labeled macromolecule which comprises carbon-13 incorporated at defined positions whereas all other carbon positions comprise carbon-12 is complexed with a ligand, wherein the macromolecule is a polypeptide, RNA, glycoprotein, protein-protein complex, protein-RNA complex, or membrane associated protein. The the complex is assessed by the perturbations in the NMR observables of the ligand as a result of the formation of the selectively labeled macromolecule in complex with the ligand using selective inversion transfer spectroscopy. The the assessments are used to derive ambiguous and non-ambiguous distance restraints used to determine the 3-dimensional structure of the the selectively labeled macromolecule in complex with the ligand.

Determining the 3 Dimensional Structure

The method for generating the 3-dimensional structure of the macromolecule in complex with a ligand demonstrated according to the present invention comprises the use of NOE's as ambiguous distance restraints in a computational docking algorithm.

Any macromolecule in which ²H, ¹³C, ¹⁵N, or ¹⁹F can be selectively incorporated at defined positions can be used in the method of the present invention. The macromolecule can be labeled with deuterium or ¹³C using any methodologies known in the art. In a preferred embodiment, the target molecule is recombinantly prepared using transformed host cells.

According to the present invention, deuterated, selectively protonated or selectively ¹³C-labeled polypeptides are prepared by transforming a host cell with an expression vector that contains a polynucleotide that encodes that polypeptide and culture the transformed cell in a deuterated culture medium that contains sources of ¹H or ¹³C that can be selectively assimilated. Sources of ¹H or ¹³C that can be selectively assimilated are well known in the art. Preferred sources are protonated or ¹³C-labeled amino acids. The preparation of deuterated culture medium is also well known in the art. Preferred culture medium contains 100% D₂O and ²H-glucose. The preparation of exemplary polypeptide target molecules that are deuterated at all positions except at defined amino acids of the macromolecule is set forth hereinafter in Example 1.

Methods for preparing expression vectors that contain polynucleotides encoding specific polypeptides are well known in the art. In a similar manner, methods for transforming host cells with those vectors and means for culturing those transformed cells so that the polypeptide is expressed are also well known in the art.

Structure determination according to the method of the present invention is accomplished by obtaining a one-dimensional magnetization transfer spectra using either saturation or inversion techniques, as is well known in the art (Mayer and Meyer, J. Am. Chem. Soc. 2001, 123, 6108-6117; Chen and Shapiro, J. Am. Chem. Soc. 2000, 122, 414-415). The NMR spectra that are preferably recorded in the method of the present invention are saturation-transfer-difference (STD) NMR spectra because of the increased sensitivity of this procedure relative to the selective inversion methods. In particular, a first free-induction decay (FID) signal is acquired in which a particular region of the protein (as opposed to the ligand) is selectively saturated using a series of selective radiofrequency pulses (typically Gaussian-shaped pulses). A second FID is then acquired in which the selective excitation is performed sufficiently off-resonance so as to perturb neither the protein nor ligand resonances. The first and second FID signals are then subtracted to yield the NOE effects. This subtraction is performed in memory during the course of the procedure. The samples are typically prepared in buffered D₂O, and a WATERGATE sequence is employed in order to facilitate suppression of the residual water signal.

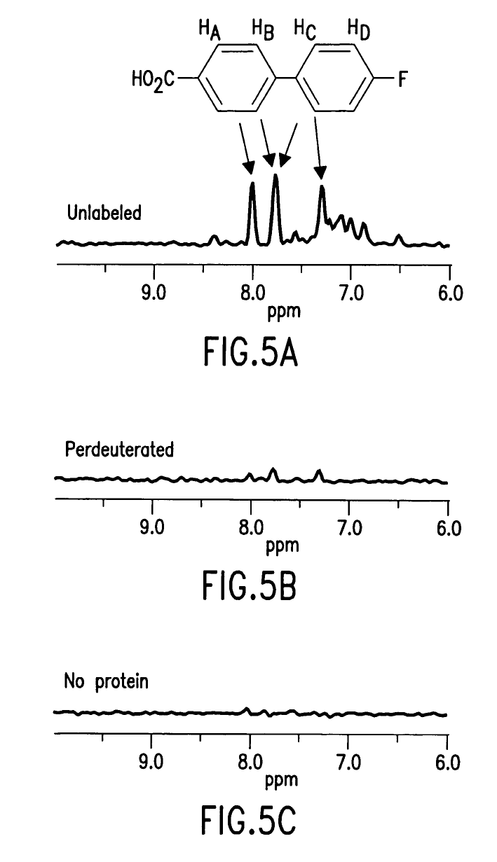

By way of example, representative STD-NMR spectra of a ligand in the absence and presence of protonated and deuterated protein are shown in FIG. 5. A detailed description of how these studies were performed can be found hereinafter in Example 2.

Distance Restraints

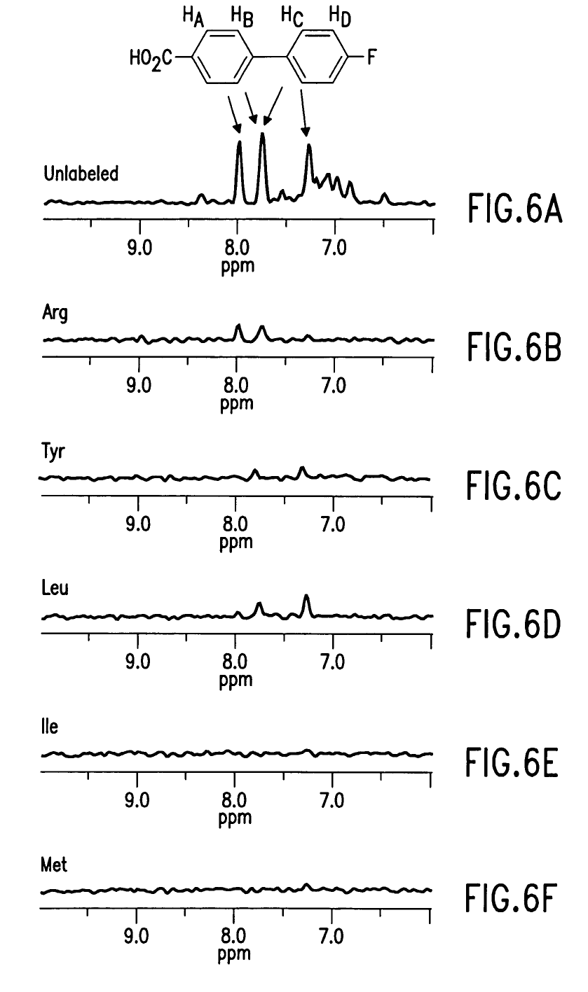

Following the determination of a series of STD-NMR spectra on a ligand in the presence of various selectively protonated samples of the macromolecule, distance restraints are derived from the data for use in the structure calculation. For each resonance of the ligand, the observed NOE intensity in the selectively protonated sample are compared to its intensity in the presence of fully protonated and fully deuterated protein. If the NOE intensity of any given ligand peak is approximately equal to that observed for the same ligand peak in the presence of deuterated protein, then the observed NOE is due to residual background protonation and not to direct contact with a site of selective protonation. Accordingly, all distances between that ligand atom and any site of selective protonation should be greater than about 5 Å. If the NOE intensity of any given ligand peak is significantly greater than that observed for the same ligand peak in the presence of deuterated protein, then the observed NOE is due to direct contact with a site of selective protonation. Accordingly, the distance between that ligand atom and at least one site of selective protonation should be less than about 5 Å. The distances can be further constrained by scaling the upper limit of the distance restraint by the observed NOE intensity, with the largest NOEs indicating a distance of less than 3 Å, medium NOEs indicating a distance of less than 4 Å, and small NOEs indicating a distance of less than 5 Å. By way of example, the STD-NMR spectra and resulting distance restraints generated for a ligand complexed to a biomolecule are given in FIG. 6 and Table 2. The determination of distance restraints is described in greater detail hereinafter in Example 2.

Following the assignment of distance restraints between the ligand atoms and the sites of protonation on the macromolecule, calculations are performed to generate structures of ligand:macromolecule complexes that are consistent with the NOE restraints. This can be accomplished with a number of commercially available software packages that currently implement or can be modified to incorporate ambiguous NOE restraints. Such packages include XPLOR (Brunger, A. T., X-PLOR Version 3.1, Yale University Press, 1992) and various docking algorithms (Ewing and Kuntz, J. Comp. Chem. 1997, 18, 1175-1189). In certain cases, the general location of the ligand binding site on the macromolecule can be determined from other methods (e.g., competitive binding experiments with a ligand whose binding site is known, chemical cross-linking experiments, etc.). This information can greatly facilitate the structure determination method of the present invention, as residues distinct from this binding site can be ignored. Although many docking algorithms require that the general location of the ligand binding site be specified, in many cases the location of the binding site on the macromolecule are nevertheless not known. According to the present invention, however, in the absence of supplementary experimental information regarding the general location of the ligand binding site, this site can be determined de novo by using the ambiguous distance restraints determined according to the present invention. For example, to determine the ligand binding site is to define Cartesian coordinates that represent possible sites of interaction on the protein surface and then weight these positions as favorable or unfavorable based on the ambiguous NOE list. This can be accomplished either by defining unique interaction points (for example, by solvating the macromolecule and using the water oxygens or by using interaction sites defined by computational modeling programs such as GRID (Goodford, J. Med. Chem. 1985, 28, 849-857)) or by predicting possible binding sites on the protein using algorithms that attempt to automatically search for ligand binding sites (such as APROPOS (Peters, et al., J. Mol. Biol. 1996, xx, 201-213) or CAST (Liang, et al., Protein Science 1998, 7, 1884-1897)). These sites can then be evaluated against the NOE data to identify the ligand binding site in a straightforward manner.

A detailed description of the binding site identification and structure determination of the complex between Bcl-xL and 4-(4-fluorophenyl)benzoic acid (3), which structure was determined using a method of the present invention, is set forth hereinafter in Example 3.

### EXAMPLES

The foregoing may be better understood by reference to the following examples which are provided for illustration and not intended to limit the scope of the present invention.

### Example 1

### Preparation of Perdeuterated, Selectively Protonated Target Molecules

A). Perdeuterated Bcl-xL

Human Bcl-xL is a member of the Bcl-2 family of proteins that are important regulators of programmed cell death. The structure of the protein is known (Muchmore, et al. Nature 1996, 381, 335-341) as well as the structure of the protein in complex with a peptide derived from Bak, a proapoptotic protein which is also a member of the Bcl-2 family of proteins (Sattler, et al. Science 1997, 275, 983-986). In the Examples of the present invention, the C-terminal histidine-tagged deletion mutant of Bcl-xL (that lacks the putative carboxy-terminal transmembrane region and residues 49-88) was used, as described previously (Sattler, et al. Science 1997, 275, 983-986). An E. Coli strain BL21 (DE3) that overexpresses the protein has already been prepared and disclosed (Sattler, et al. Science 1997, 275, 983-986).

Perdeutered Bcl-xL was prepared by growing the E. Coli strain that overexpresses Bcl-xL in a culture medium comprised of 7.48 g of anhydrous Na₂HPO₄, 3.61 g of anhydrous KH₂PO₄, 0.58 g of NaCl, 1.2 mL of a 100 mM solution of CaCl₂ (dissolved in D₂O), 1.2 mL of a 0.5% solution of thiamine-HCl (dissolved in D₂O), 1.2 mL of a 1 M solution of MgSO₄ (dissolved in D₂O), 30 mg of kanamycin (Sigma), 2 g of U-²H-glucose (Cambridge Isotope Laboratories, CIL) and 1 g of NH₄Cl (CIL) dissolved in 1 liter of D₂O (CIL). The medium was sterilized via filtration using a 0.2 micron filter (Millipore) and transferred to a flask. The flask contents were then inoculated with 1 mL of glycerol stock of genetically-modified E. Coli strain BL21(DE3). The flask contents were shaken (225 rpm) at 37° C. until an optical density of 0.8 was observed. The cells were then induced by adding 1 mL of a 1 M solution of IPTG to the growth medium and allowed to shake at 37° C. for an additional 3 hours. The cells were harvested by centrifugation at 17,000×g for 10 minutes at 4° C. and the resulting cell pellets were collected and stored at −85° C. The wet cell yield was 5 g/L. Analysis of the soluble and insoluble fractions of cell lysates by sodium dodecyl sulfate polyacrylamide gel electrophoresis (SDS-PAGE) revealed that greater than 50% of the Bcl-xL was found in the soluble phase.

The Bcl-xL protein was purified by affinity chromatography on Ni-NTA resin. The cell pellets were resuspended a buffer containing 10 mM NaPO₄, 150 mM NaCl, pH 7.4. The cells were then lysed using a microfluidizer (Microfluidics International), and the resulting lystate clarified via centrifugation at 76,000×g for 10 minutes. The clarified cell lysate was loaded onto a column containing Ni-NTA resin (Novagen). The resin was washed with 10 column volumes of a buffer containing 10 mM NaPO₄, 150 mM NaCl, 25 mM imidazole, pH 7.4. The protein was eluted with 5 column volumes of a buffer containing 10 mM NaPO₄, 150 mM NaCl, 500 mM imidazole, pH 7.4. The final eluent was concentrated using centriprep concentrators (Millipore) and dialyzed in D₂O buffer containing 20 mM Na₂PO₄, pH 7.0 overnight to provide approximately 10 mgs of protein per liter of culture medium.

Perdeuterated Bcl-xL prepared according to this procedure will hereinafter be designated as U-²H-Bcl-xL. A ¹H-NMR spectrum of U-²H-Bcl-xL is shown in FIG. 2B.

B). Perdeuterated, ¹H-Leu-Bcl-xL

Perdeuterated Bcl-xL that is selectively protonated at all leucine residues was prepared as described above except for the addition of 50 mgs of leucine (Sigma) per liter of culture medium. Perdeuterated, selectively leucine protonated Bcl-xL prepared according to this procedure will hereinafter be designated as U-²H-{¹H-L}-Bcl-xL. A ¹H-NMR spectrum of U-²H-{¹H-L}-Bcl-xL is shown in FIG. 2C.

C). Perdeuterated, ¹H-Ile-Bcl-xL

Perdeuterated Bcl-xL that is selectively protonated at all isoleucine residues was prepared as described above except for the addition of 50 mgs of isoleucine (Sigma) per liter of culture medium. Perdeuterated, selectively isoleucine protonated Bcl-xL prepared according to this procedure will hereinafter be designated as U-²H-{¹H-I}-Bcl-xL. A ¹H-NMR spectrum of U-²H-{¹H-I}-Bcl-xL is shown in FIG. 2D.

D). Perdeuterated, ¹H-Arg-Bcl-xL

Perdeuterated Bcl-xL that is selectively protonated at all arginine residues was prepared as described above except for the addition of 50 mgs of arginine (Sigma) per liter of culture medium. Perdeuterated, selectively arginine protonated Bcl-xL prepared according to this procedure will hereinafter be designated as U-²H-{¹H-R}-Bcl-xL. A ¹H-NMR spectrum of U-²H-{¹H-R}-Bcl-xL is shown in FIG. 2E.

E). Perdeuterated, ¹H-Met-Bcl-xL

Perdeuterated Bcl-xL that is selectively protonated at all methionine residues was prepared as described above except for the addition of 50 mgs of methionine (Sigma) per liter of culture medium. Perdeuterated, selectively methionine protonated Bcl-xL prepared according to this procedure will hereinafter be designated as U-²H-{¹H-M}-Bcl-xL. A ¹H-NMR spectrum of U-²H-{¹H-M}-Bcl-xL is shown in FIG. 2F.

F). Perdeuterated, ¹H-Tyr-Bcl-xL

Perdeuterated Bcl-xL that is selectively protonated at all tyrosine residues was prepared as described above except for the addition of 50 mgs of tyrosine (Sigma) per liter of culture medium. Perdeuterated, selectively tyrosine protonated Bcl-xL prepared according to this procedure will hereinafter be designated as U-²H-{¹H-Y}-Bcl-xL.

G). Unlabeled Bcl-xL

Bcl-xL that is unlabeled was prepared as described above except that unlabeled glucose (Sigma) was used in place of U-²H-glucose and the medium was dissolved in 1 L of distilled water as opposed to D₂O. Bcl-xL prepared according to this procedure will hereinafter be designated as unlabeled Bcl-xL. A ¹H-NMR spectrum of unlabeled Bcl-xL is shown in FIG. 2A.

### Example 2

### Acquisition and Analysis of STD-NMR Spectra on Bcl-xL/Ligand Complexes

A sample of unlabeled Bcl-xL as well as samples of perdeuterated Bcl-xL that were selectively protonated at the specific amino acids Arg, Tyr, Leu, Ile, and Met were prepared in accordance with the procedures of Example 1. Samples of 200 uM 4-(4-fluorophenyl)benzoic acid (3, Array),

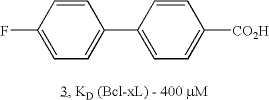

which was independently determined to be a ligand for Bcl-xL using NMR-based screening, were prepared in a 100% D₂O buffer comprised of 25 mM Na₂PO₄, 5% DMSO-d₆ (Aldrich), pH 7.0. 500 uL of the ligand solution was transferred to each of 8 NMR tubes (Wilmad). The protein solutions were then added to the NMR tubes to a final protein concentration of 40 uM in the following manner: Sample 1: unlabeled Bcl-xL; Sample 2: U-²H-Bcl-xL; Sample 3: U-²H-{¹H-R}-Bcl-xL; Sample 4: U-²H-{¹H-Y}-Bcl-xL; Sample 5: U-²H-{¹H-L}-Bcl-xL; Sample 6: U-2H-{¹H-I}-Bcl-xL; Sample 7: U-²H-{¹H-M}-Bcl-xL; Sample 8: No protein.

STD-NMR spectra (Mayer and Meyer, J. Am. Chem. Soc. 2001, 123, 6108-6117) were determined on each of the eight samples. All NMR experiments were performed at 300K on a Bruker Avance DRX500 system equipped with a CryoProbe. The pulse sequence for the ID STD-NMR spectra was identical to that used by Mayer and Meyer (2001) except that a CPMG T₂-filter was used instead of a T₁ₚ-filter to remove background protein resonances. Subtraction of the on- and off-resonance spectra was performed internally and a WATERGATE sequence was used for suppression of the residual solvent signal. On-resonance irradiation of the protein was performed by using a train of Gauss-shaped pulses of 2.5 ms length separated by a 2.5 ms delay, and alternating on-resonance irradiation was applied at 0.8 and 3.0 ppm. Off-resonance irradiation was applied at −10.0 ppm. 280 shaped pulses were used in the pulse train, leading to a total saturation time of 1.4 s. All spectra were acquired with a 100 ms T₂-filter, 1024 complex points, 1024 scans, a ¹H sweep width of 8333 Hz, and a total recycle time of 2.0 s.

All NMR spectra were processed and analyzed on Silicon Graphics computers using standard protocols well-known in the art. STD-NMR spectra for biaryl 3 in the presence of unlabeled Bcl-xL (Sample 1), U-²H-Bcl-xL (Sample 2), and no protein are shown in FIG. 5. The intensity observed in the STD-NMR spectrum for the ligand in the presence of U-²H-Bcl-xL are significantly reduced compared to unlabeled protein but not eliminated due to residual protonation of the protein. Therefore, the signal intensity for each resonance of the biaryl 3 in the presence of unlabeled and perdeuterated protein was taken as the maximum and minimum intensity, respectively, for the NOE in the presence of the selectively protonated samples. The intensity of the NOEs could then be calculated using the following equation,

$$I ( norm ) = \frac{I ( S L ) - I ( {}^{2} H )}{I ( {}^{1} H ) - I ( {}^{2} H )}$$

where I(norm) is the normalized intensity in the STD-NMR spectrum for a given biaryl resonance, and I(SL), I(²H), and I(¹H) are the intensities of the resonance in the presence of the selectively protonated, perdeuterated, and unlabeled protein, respectively. The results of this analysis for biaryl 3 and Bcl-xL are given in Table 1. The normalized NOE intensities can be visualized by simple subtraction of the STD-NMR spectrum of the ligand in the presence of deuterated protein from each of the selectively protonated samples. The results of this spectral subtraction procedure for biaryl 3 and Bcl-xL are shown in FIG. 6. In these spectra, it is clearly seen that an arginine residue is in close contact with the benzoic acid portion of 3 (magnetization transfer to H_{A} and H_{B/C}), but not the fluorophenyl portion. It is also clear that a tyrosine and leucine residue is in contact with the fluorophenyl portion of 3 (magnetization transfer to H_{D} and H_{B/C}), but not the benzoic acid portion. Spectra shown in (E) and (F) indicate that no portion of the biaryl is in contact with any isoleucine or methionine residues.

TABLE 1
NOE intensities

| | Unlabeled | U-²H- | U-²H-{¹H-R}- | U-²H-{¹H-Y}- | U-²H-{¹H-L}- | U-²H-{¹H-I}- | U-²H-{¹H-M}- |
|---|---|---|---|---|---|---|---|
| Sample/ | Bcl-xL | Bcl-xL | Bcl-xL | Bcl-xL | Bcl-xL | Bcl-xL | Bcl-xL |
| Proton | 1 | 2 | 3 | 4 | 5 | 6 | 7 |
| HA | 100^{a} | 0 | 25 | 3 | 8 | 1 | 4 |
| | (175) | (17) | (57) | (22) | (28) | (18) | (23) |
| HB/HC | 100 | 0 | 23 | 10 | 23 | 0 | 0 |
| | (200) | (32) | (70) | (49) | (71) | (30) | (30) |
| HD | 100 | 0 | 9 | 22 | 47 | 9 | 12 |
| | (167) | (28) | (40) | (59) | (93) | (40) | (45) |

^{a}Normalized NOE intensity. Actual NOE intensity is shown in parentheses.

These NOE intensities were then converted into distance restraints for use in structural calculations in the following manner. For any given amino acid type and ligand resonance, normalized NOE intensities less than 5% were treated as having no interaction. Therefore, distances between that ligand atom and all protons on all amino acids of that specific type were set at greater than 5 Å. For NOE intensities greater than 15%, ambiguous distance restraints were assigned. Normalized NOE intensities between 15 and 25% were treated as weak interactions, and at least one distance between that ligand atom and at least one proton from an amino acid of that specific type should be less than 5 Å. Normalized NOE intensities between 25 and 45% were treated as moderate interactions, and at least one distance between that ligand atom and at least one proton from an amino acid of that specific type should be less than 4 Å. Normalized NOE intensities greater than 45% were treated as strong interactions, and at least one distance between that ligand atom and at least one proton from an amino acid of that specific type should be less than 3 Å. The results of this analysis on the five selectively protonated samples of Bcl-xL resulted in a total of 10 ambiguous NOEs and are shown in Table 2.

TABLE 2
Distance Restraints

| | U-²H-{¹H-R}- | U-²H-{¹H-Y}- | U-²H-{¹H-L}- | U-²H-{¹H-I}- | U-²H-{¹H-M}- |
|---|---|---|---|---|---|
| | Bcl-xL | Bcl-xL | Bcl-xL | Bcl-xL | Bcl-xL |
| Sample | 3 | 4 | 5 | 6 | 7 |
| HA | <4^{a} | >5 | — | >5 | >5 |
| | (25) | (3) | (8) | (1) | (4) |
| HB/HC | <5 | — | <5 | >5 | >5 |
| | (23) | (10) | (23) | (0) | (0) |
| HD | —^{b} | <5 | <3 | — | — |
| | (9) | (22) | (47) | (9) | (12) |

^{a}Distance restraint (in Å) derived from the NOE intensity data. Normalized NOE intensity is shown in parentheses.
^{b}Peaks with normalized NOE intensities in the range of 5-15% were not used to assign a distance restraint since this close to the experimental error.

As an example of the use of distance restraints that define no interaction and those that define ambiguous interactions, consider the NOE intensities for biaryl 3 in the presence of U-²H-{¹H-Y}-Bcl-xL. In this case, the HA protons of the ligand have a normalized NOE intensity of 3%, indicating no interaction with tyrosine residues. Therefore, the distance restraint list should reflect that both of the HA protons on biaryl 3 are more distant than 5 Å from the Hα, Hβ, Hδ, and Hε protons of every tyrosine in the protein (in this case Y19, Y26, Y105, Y124, Y177, and Y199). In contrast, the HD protons of the ligand have a normalized NOE intensity of 22%, indicating a weak interaction with at least one proton of one tyrosine residue. Therefore, the distance restraint list should reflect that at least one of the HD protons on biaryl 3 is within 5 Å of an Hα, Hβ, Hδ, or Hε proton of any tyrosine in the protein (in this case Y19, Y26, Y105, Y124, Y177, or Y199).

### Example 3

### Structure Determination of a Bcl-xL/Ligand Complex Using Ambiguous NOEs Derived from Selectively Labeled Samples

Ambiguous distance restraints for biaryl 3 in complex with Bcl-xL were obtained for arginine, tyrosine, leucine, isoleucine, and methionine labeled samples as described above. Once a distance restraint list has been generated, structure calculations can be performed. For the Bcl-xL/biaryl 3 complex, DOCK4.0 (Kuntz, et al., J. Mol. Biol. 1982, 161, 269-288) was used. When using docking algorithms such as DOCK, the structure of the macromolecule and the general ligand binding site on the macromolecule need to be identified. This was accomplished by taking the known structure of Bcl-xL complexed to biaryl 3 (see Example 4), solvating the protein using the program InsightII (Biosym), and then evaluating the solvent molecules for compatibility with the ambiguous distance restraints in order to define potential binding sites to use in DOCK4.0.

A total of 1554 solvent molecules were added to Bcl-xL using the SOAK command within InsightII. Next, each of the water oxygen positions was treated as a ligand mimic and assigned an NOE penalty (E_{NOE}) based on the ambiguous NOE list derived for biaryl 3. This was performed for each ambiguous NOE by finding the closest atom of the corresponding residue type to the solvent position, and then calculating an NOE penalty according to the following equation:

$$\begin{matrix}If ( {R}_{min}^{′} < {R}_{lower}^{′} ) \\ If ( {R}_{min}^{′} > {R}_{upper}^{′} )\end{matrix} \{ \begin{matrix}{E}_{i} = {( {R}_{min}^{′} - {R}_{lower}^{′} )}^{2} \\ {E}_{i} = {( {R}_{min}^{′} - {R}_{upper}^{′} )}^{2}\end{matrix} \}$$

where Rⁱₘᵢₙ is the closest atom of the corresponding residue type for the ith NOE, Rⁱ_{lower} and Rⁱᵤₚₚₑᵣ are the lower and upper bounds, respectively, for the ith NOE, and Eᵢ is the energy penalty associated with the ith NOE. The total NOE penalty (E_{NOE}) for each solvent position is then the sum of each penalty Eᵢ over all NOEs.

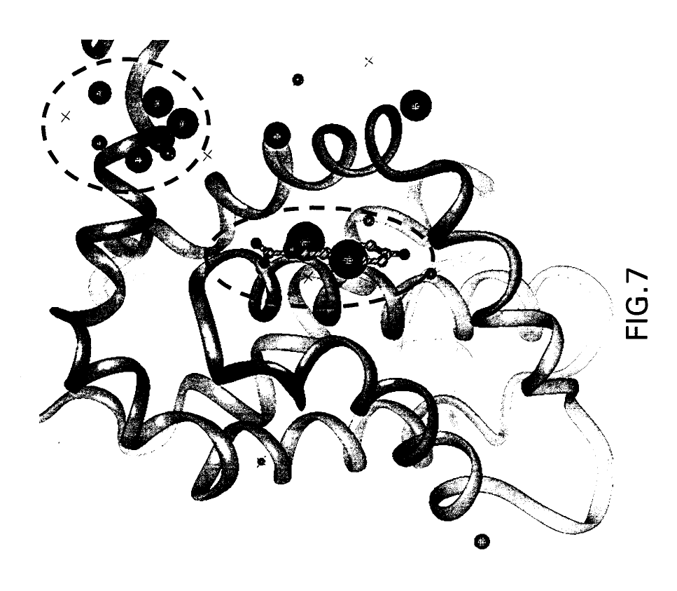

The results of this analysis on Bcl-xL using the NOE list derived for biaryl 3 is shown graphically in FIG. 7. In this figure, the radius of each solvent was scaled according to its energy penalty, with the lowest energy having the largest radii. It was determined from this analysis that there are two major clusters of solvent positions that can most favorably fulfill the ambiguous NOE list derived for biaryl 3. These two clusters both occur along a continuous hydrophobic groove on the protein surface that is known to be the interaction site for peptides and proteins (Sattler, et al. Science 1997).

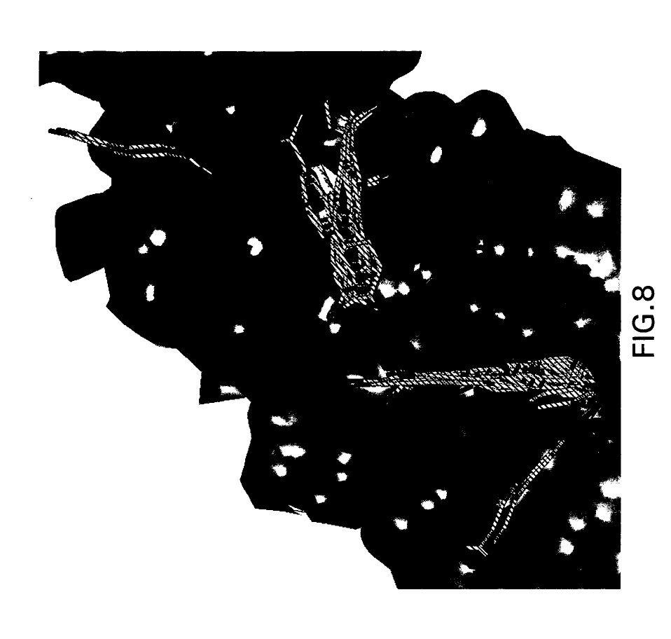

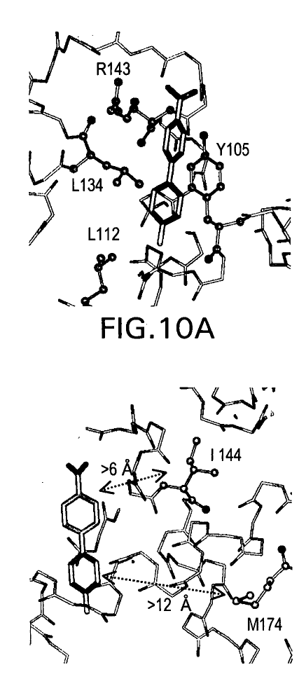

After identifying these two solvent clusters, DOCK4.0 was run using an interaction surface that encompassed both of the potential sites. Dock was run within InsightII in single mode using default parameters, using the protein structure of Bcl-xl when complexed to biaryl 3 (see Example 4). A total of 35 low-energy conformations were identified by the docking algorithm, and these are shown in FIG. 8. All conformations have DOCK4.0 energies less than −9.0 units, with an average of −16.5±3.8 units (range −9.32 to −21.20). Next, the 34 structures predicted by the computational algorithm were assigned an NOE penalty according to the equation given above. A total of 11 conformations of biaryl 3 were allowed by the ambiguous NOE data (E_{NOE}<1.0), and all of these conformations are in agreement with the high-resolution structure of the Bcl-xL/3 complex. As shown in this figure, the fluorophenyl portion of ligand 3 is in contact with Tyr105 (yellow carbon atoms), Leu 134 (yellow carbon atoms), and L112 (yellow carbon atoms) of Bcl-xL, while the benzoic acid portion of 3 is in contact with Arg143 (yellow carbon atoms) of Bcl-xL. As shown in FIG. 10B, no protons on 3 are within 5 Å of any proton on an isoleucine or methionine residue, as the closets isoleucine is >6 Å away (Ile144, yellow carbon atoms) and the closest methionine is >12 Å away (Met174, yellow carbon atoms).

### Example 4

### Conventional, High-Resolution NMR Structure Determination of a Bcl-xL/Ligand Complex

For comparison to the structure of the Bcl-xL/biaryl 3 complex determined using the present invention, a high-resolution NMR structure of the biaryl 3 complexed to Bcl-xL was obtained using conventional techniques.

The cloning, expression, and purification of the catalytic domain of Bcl-xL uniformly labeled with ¹⁵N (1 g/L¹⁵NH₄Cl as the sole nitrogen source) and ¹³C (2 g/L ¹³C-glucose as the sole carbon source) was accomplished using the procedures set forth in Example 1. The NMR samples consisted of 1.0 mM ¹⁵N, ¹³C-Bcl-xL and 1.0 mM biaryl 3 in a 10% D₂O buffer containing 25 mM sodium phosphate (pH 7.0).

The ¹H, ¹³C, and ¹⁵N resonances of Bcl-xL in complex with biaryl 3 were assigned from a comparison of ¹³C-separated 3D NOESY-HMQC (S. Fesik, et al., J. Magn. Reson, 87: 588-593 (1988)); D. Marion, et al., J. Am. Chem. Soc, 111: 1515-1517 (1989)) and ¹⁵N-separated 3D NOESY-HSQC spectra of the Bcl-xL/3 complex to those of the apo protein, for which the assignments are known (Muchmore, et al. Nature 1996, 381, 335-341). In this way, >95% of all residues were assigned.

To detect NOEs between the biaryl 3 and Bcl-xL, a 3D ¹²C-filtered, ¹³C-edited NOESY spectrum was collected. The pulse scheme consisted of a double ¹³C-filter sequence (A. Gemmeker, et al., J. Magn. Reson, 96: 199-204 (1992)) concatenated with a NOESY-HMQC sequence (S. Fesik, et al., J. Magn. Reson., 87: 588-593 (1988)); D. Marion, et al., J. Am. Chem. Soc, 111: 1515-1517 (1989)). The spectrum was recorded with a mixing time of 80 ms.

To identify amide groups that exchanged slowly with the solvent, a series of ¹H, ¹⁵N-HSQC spectra (G. Bodenhausen, et al., J. Chem. Phys. Lett., 69: 185-189 (1980)) were recorded at 25° C. at 2 hr intervals after the protein was exchanged into D₂O. The acquisition of the first HSQC spectrum was started 2 hrs. after the addition of D₂O.

All NMR spectra were recorded at 30° C. on a Bruker AMX500 or AMX600 NMR spectrometer. The NMR data were processed and analyzed on Silicon Graphics computers. In all NMR experiments, pulsed field gradients were applied where appropriate as described (A. Bax, et al., J. Magn. Reson, 99: 638 (1992)) to afford the suppression of the solvent signal and spectral artifacts. Quadrature detection in indirectly detected dimensions was accomplished by using the States-TPPI method (D. Marion, et al., J. Am. Chem. Soc., 111: 1515-1517 (1989)). Linear prediction was employed as described (E. Olejniczak, et al., J. Magn. Reson., 87: 628-632 (1990)).

Distance restraints derived from the NOE data were classified into three categories based on the NOE cross peak intensity and given a lower bound of 1.8 Å and upper bounds of 3.0 Å, 4.0 Å, and 5.0 Å, respectively. Hydrogen bonds, identified for slowly exchanging amides based on initial structures, were defined by two restraints: 1.8-2.5 Å for the H—O distance and 1.8-3.3 Å for the N—O distance. Structures were calculated with the X-PLOR 3.1 program (A. Brunger, “XPLOR 3.1 Manual,” Yale University Press, New Haven, 1992) on Silicon Graphics computers using a hybrid distance geometry-simulated annealing approach (M. Nilges, et al., FEBS Lett., 229: 317-324 (1988)).

A total of 1843 approximate interproton distance restraints were derived from the NOE data. In addition, 48 unambiguous intermolecular distance restraints between Bcl-xL and biaryl 3 were derived from a 3D ¹²C-filtered, ¹³C-edited NOESY spectrum. The experimental restraints also included 114 distance restraints corresponding to 57 hydrogen bonds. The amides involved in hydrogen bonds were identified based on their characteristically slow exchange rate, and the hydrogen bond partners from initial NMR structures calculated without the hydrogen bond restraints. The total number of non-redundant, experimentally-derived restraints was 2005.

The structures were consistent with the NMR experimental restraints. There were no distance violations greater than 0.4 Å. In addition, the simulated energy for the van der Waals repulsion term was small, indicating that the structures were devoid of inferior inter-atomic contacts. The NMR structures also exhibited good covalent bond geometry, as indicated by small bond-length and bond-angle deviations from the corresponding idealized parameters. The average atomic root mean square deviation of the 8 structures for residues 5-35 and 90-200 from the mean coordinates was 0.88 Å for backbone atoms (C^{α}, N, and C′), and 3.22 Å for all non-hydrogen atoms.

The structure indicates that the biaryl 3 is binding in the hydrophobic groove that forms the peptide-binding site (Sattler, et al. Science 1997, 275, 983-986). The biaryl interacts with Tyr105, Arg143, Leu112, Leu134, Ala146, Ala108, Phe101, and Phe109. No isoleucine or methionine residues are in the binding pocket, consistent with the NOE data of the present invention (see FIG. 10).

### Example 5

### Structure Determination of a Bcl-xL/Ligand Complex Ambiguous NOEs Derived from Selectively Labeled Samples

As set forth in Example 3 above, the method of the present invention can define the structure of a protein-ligand complex from ambiguous distance restraints when the conformation of the protein when complexed to the ligand of interest is known. However, it is typically the case that the specific conformation of the protein when complexed to the ligand of interest is not known. Under such circumstances, the structure of the protein alone or when complexed to some other molecule should be used as a proxy in the docking simulations of the present invention.

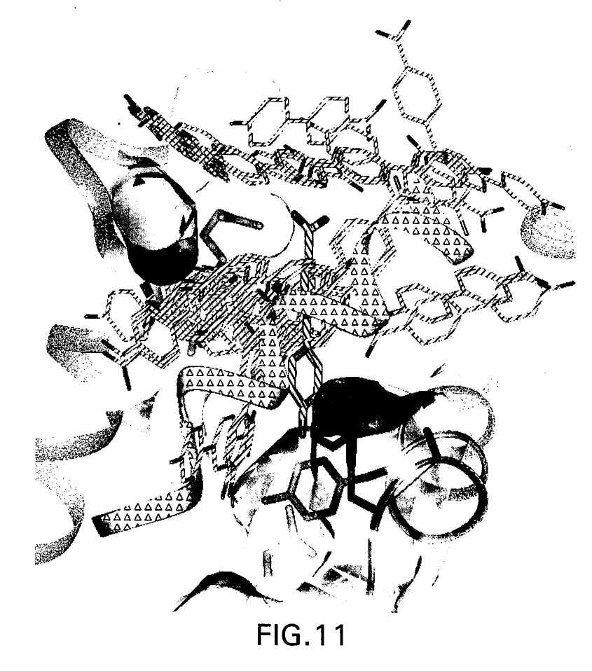

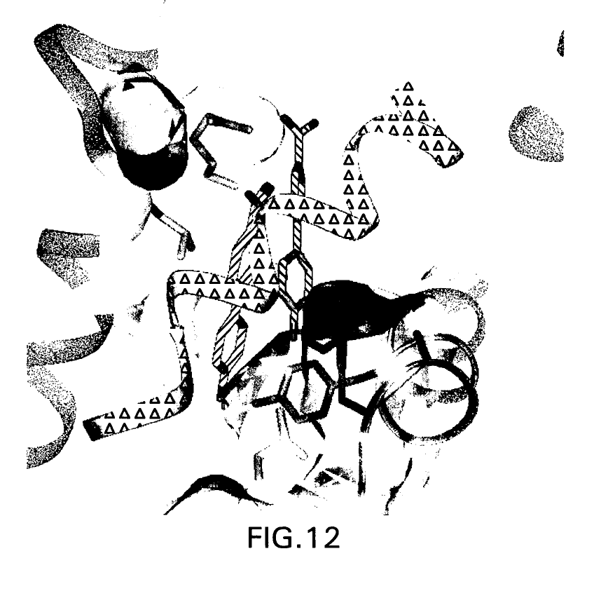

As an example, the same ambiguous NOE restraint list as defined in Example 2 was used to determine the structure of biaryl 3 in complex with Bcl-xL when using the conformation of Bcl-xL complexed to the Bak peptide (Sattler, et al. Science 1997, 275, 983-986). As in Example 3, the binding site was identified by weighting solvent atoms against the ambiguous NOE restraint list. The same binding site on Bcl-xL was identified as in Example 3, and DOCK4.0 was used to dock biaryl 3 to the protein. As shown in FIG. 11, a total of 45 conformations were found by the docking algorithm with energies less than 0.0 units (average energy of −2.28±1.38). Next, the 45 structures predicted by the computational algorithm were assigned an NOE penalty according to the equation given above. A total of 2 conformations of biaryl 3 were allowed by the ambiguous NOE data (E_{NOE}<0.1), both of which place the biaryl 3 in the correct orientation in the binding pocket. FIG. 12 depicts the orientations of biaryl 3 predicted by the computational algorithm DOCK4.0 that are allowed by the ambiguous NOE data of the current invention (E_{NOE}<0.1, yellow carbon atoms) compared to orientation derived from traditional high-resolution NMR spectroscopy (green carbon atoms). Despite the large conformational differences in Bcl-xL that exist when complexed to the Bak peptide vs. biaryl 3, the docking method of the present invention correctly identified the binding site as well as the orientation of the biaryl ligand.

The foregoing examples are presented for purposes of illustration and are not intended to limit the scope of the invention. Accordingly, variations and changes as would be understood by one skilled in the art are intended to be within the scope and nature of the invention as defined in the appended claims.

What is claimed is:

1. A method of determining the 3-dimensional structure of a macromolecule in complex with a ligand, said method comprising the steps of:
a). forming a complex comprising a macromolecule and a ligand by exposing a selectively labeled macromolecule with a ligand molecule that forms a complex with said macromolecule;
b). determining one or more portions of said ligand that are in close proximity to the selectively labeled regions of the macromolecule in said complex;
c). deriving ambiguous and non-ambiguous distance restraints between said selectively labeled macromolecule and said ligand molecule in said complex from step b;
d). determining the 3-dimensional structure of said macromolecule in said complex using said ambiguous and non-ambiguous distance restraints.

2. The method according to claim 1, wherein
said macromolecule in complex with said ligand is selected from the group consisting of a polypeptide, RNA, glycoprotein, protein-protein complex, protein-RNA complex, membrane associated protein.

3. The method according to claim 1, wherein said selectively labeled macromolecule comprises ¹H at defined positions, whereas all other proton positions comprise ²H.

4. The method according to claim 3, wherein
said portions of said ligands are assessed by the perturbations in NMR observables of the ligand as a result of formation of said selectively labeled macromolecule in complex with said ligand.

5. The method according to claim 4 wherein
said assessment is saturation transfer difference spectroscopy.

6. The method according to claim 5, wherein
said macromolecule in complex with said ligand is a polypeptide.

7. The method of claim 1 wherein said macromolecule comprises carbon-13 nuclei at defined positions, whereas all other carbon positions comprise carbon-12.

8. The method according to claim 7 wherein
said portions of said ligands are assessed by the perturbations in NMR observables of the ligand as a result of formation of said macromolecule in complex with said ligand.

9. The method of claim 8 wherein
said assessment is selective inversion transfer methods.

10. The method according to claim 9, wherein
said macromolecule in complex with said ligand is a polypeptide.
